(19) United States
(12) Patent Application Publication
(10) Pub. No.: US 2010/0076240 A1
(43) Pub. Date: Mar. 25, 2010

(54) Production of Olefins

(75) Inventors: Jean-Pierre Dath, Beloeil Hainault (BE); Walter Vermeiren, Houthalen (BE)

Correspondence Address: FINA TECHNOLOGY INC, PO BOX 674412, HOUSTON, TX, 77267-4412, US

(73) Assignee: TOTAL PETROCHEMICALS RESEARCH FELUY, SENEFFE (BE)

(21) Appl. No.: 12/374,811

(22) PCT Filed: Jul. 13, 2007
(86) PCT No.: PCT/EP07/57260

§ 371 Date: Sep. 2, 2009

(30) Foreign Application Priority Data
Jul. 26, 2006 (EP) 06117863.8

(51) Int. Cl. C07C 4/06

(52) U.S. Cl. 585/653

(57) ABSTRACT

A process for converting a hydrocarbon feedstock to provide an effluent containing light olefins, the process comprising passing a hydrocarbon feedstock, the feedstock containing at least 25 wt % C₅₊ paraffins, through a reactor containing a crystalline silicate catalyst to produce an effluent including propylene.

The present invention relates to a process for converting a paraffin-containing hydrocarbon feedstock to produce an effluent containing light olefins, in particular propylene.

There is an increasing demand for light olefins, for example ethylene and propylene, in the petrochemical industry, in particular for the production of polymers, in particular polyethylene and polypropylene. In particular, propylene has become an increasingly valuable product and accordingly there has been a need for the conversion of various hydrocarbon feedstocks to produce propylene.

It has been known for a number of years to convert paraffins into light olefins, with the effluent containing ethylene and propylene. Conventionally, paraffins are thermally cracked at high temperatures (greater than 750° C.) in the presence of steam. The primary product in the effluent is ethylene, and the secondary important product is propylene, followed by heavier hydrocarbons that are very rich in multi-unsaturated products, such as dienes. It is not possible to alter the steam cracking process in order to obtain propylene as the major product. Moreover, the heavier diene-rich cuts have to be treated for further valorisation.

It is an object of the present invention to provide a process for converting a paraffin-containing hydrocarbon feedstock to produce an effluent containing light olefins, in particular propylene.

It is another object of the invention to provide a process for producing propylene having a high propylene yield and purity.

It is a further object of the present invention to provide such a process, which can produce olefin effluents from which propylene can readily be cut.

It is yet a further object of the present invention to provide a process for producing an effluent containing light olefins, in particular propylene, having a stable conversion and a stable product distribution over time.

The present invention provides a process for converting a hydrocarbon feedstock to provide an effluent containing light olefins, the process comprising passing a hydrocarbon feedstock, the feedstock containing at least 25 wt % C₅₊ paraffins, through a reactor containing a crystalline silicate catalyst to produce an effluent including propylene.

Preferably, the method further comprises the step of forming the hydrocarbon feedstock by adding at least one C₆₊ paraffin, preferably linear, to a C₄+ hydrocarbon feedstock cut comprising C₄₊ paraffins.

More preferably, the at least one C₆₊ paraffin comprises at least one C₆₋₂₀ linear paraffin.

More preferably, the hydrocarbon feedstock comprises from 1 to 80 wt % of the at least one C₆₊ linear paraffin and 20 to 99 wt % of the C₄+ hydrocarbon feedstock cut comprising C₄₊ paraffins.

Preferably, the hydrocarbon feedstock contains at least one C₄₊ olefin.

The C₄+ hydrocarbon feedstock cut comprising C₄₊ paraffins, comprises a blend of C4 cuts (like hydrogenated raw C₄'s, raffinate I and raffinate II) and cracked gasoline cuts originating from a FCC, coker or visbreaker unit.

Preferably, the crystalline silicate is an MFI-type crystalline silicate having a silicon/aluminium atomic ratio of from 120 to 1000.

Preferably, the MFI-type crystalline silicate catalyst comprises silicalite.

Preferably, the hydrocarbon feedstock is passed over the crystalline silicate at a reactor inlet temperature of from 500 to 600° C., more preferably from 550 to 600° C., most preferably about 575° C.

Preferably, the hydrocarbon feedstock is passed over the crystalline silicate at a liquid hourly space velocity (LHSV) of from 5 to 30 h⁻¹, more preferably from 5 to 15 h⁻¹.

Preferably, the hydrocarbon feedstock is passed over the crystalline silicate at a pressure of from 0 to 2 bara, more preferably from 1 to 2 bara, most preferably about 1.5 bara.

The present invention can thus provide a process wherein paraffin-containing streams (products) from refinery and petrochemical plants are selectively converted not only into light olefins, but particularly into propylene. The streams may contain olefins which are also converted into light olefins such as propylene. Linear C₅ to C₂₀, in particular C₆₊, paraffins are particularly converted into light olefins such as propylene.

The various aspects of embodiments of the present invention will now be described in greater detail, by way of example only, with reference to the accompanying drawings, in which:

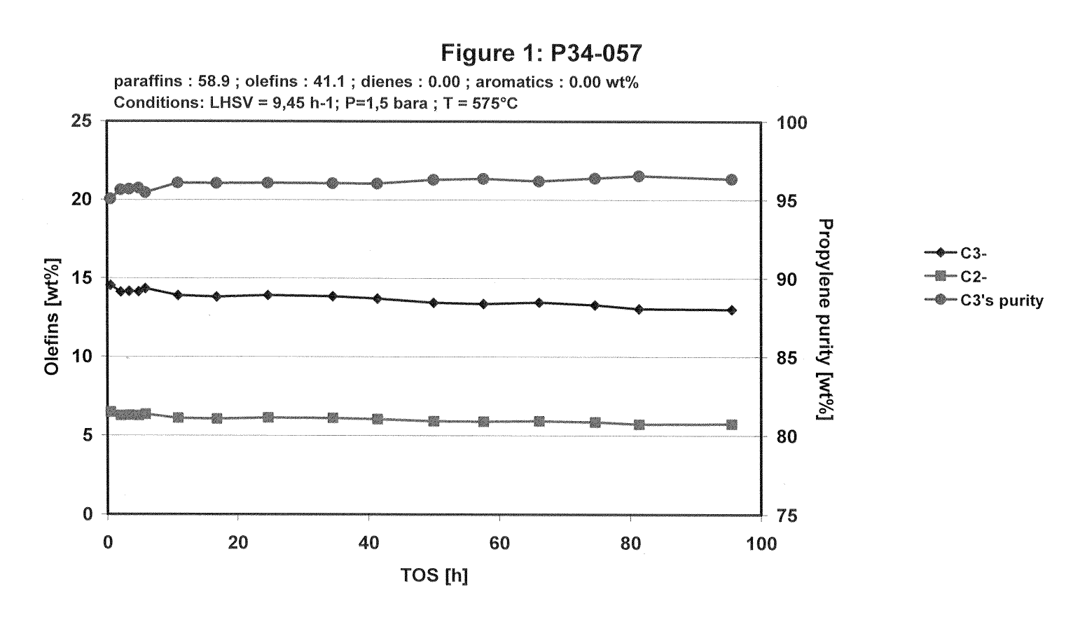

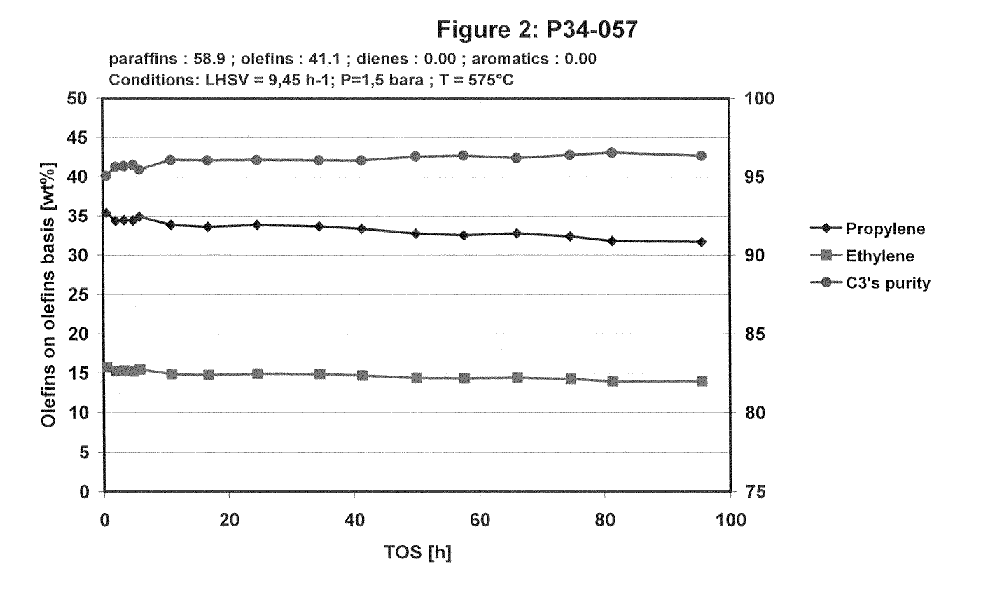

FIGS. 1 and 2 show the relationship between the olefins in the effluent and the propylene purity with respect to the time on stream (TOS) (FIG. 1) and the relationship between the olefin yield on an olefins basis and the time on stream (TOS) (FIG. 2) for Example 1 of the invention;

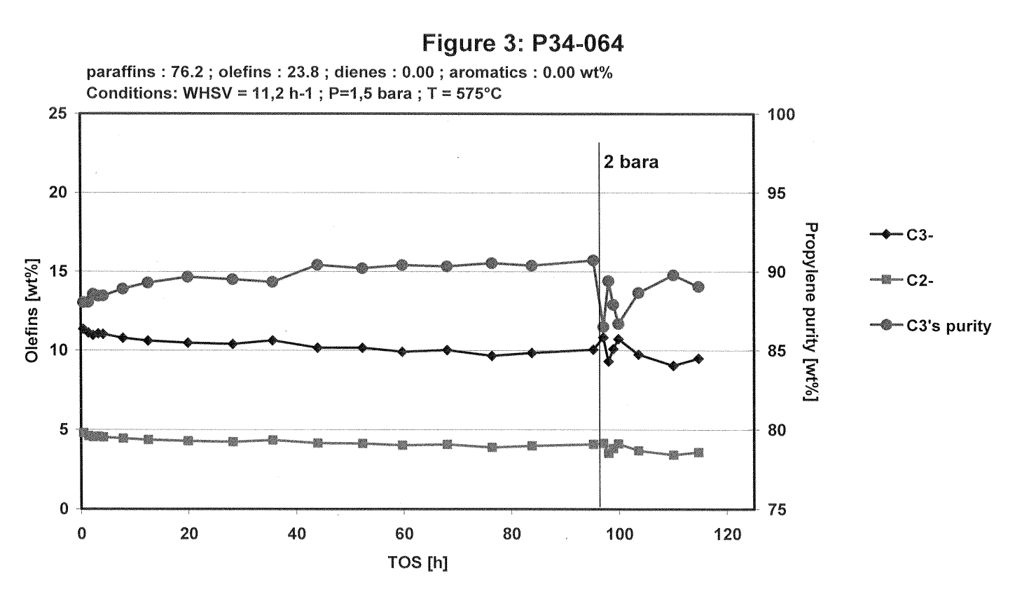

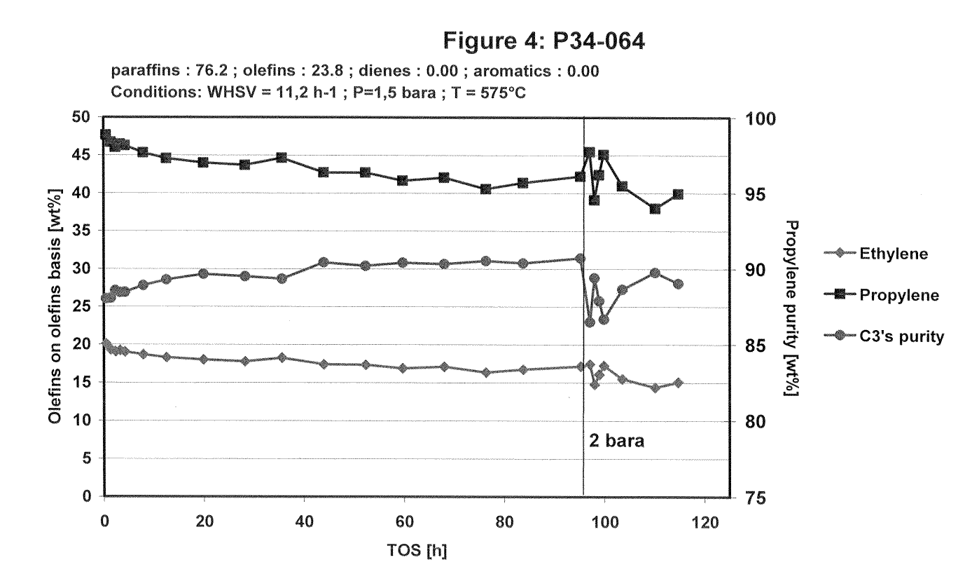

FIGS. 3 and 4 show the relationship between the olefins in the effluent and the propylene purity with respect to the time on stream (TOS) (FIG. 3) and the relationship between the olefin yield on an olefins basis and the time on stream (TOS) (FIG. 4) for Example 2 of the invention;

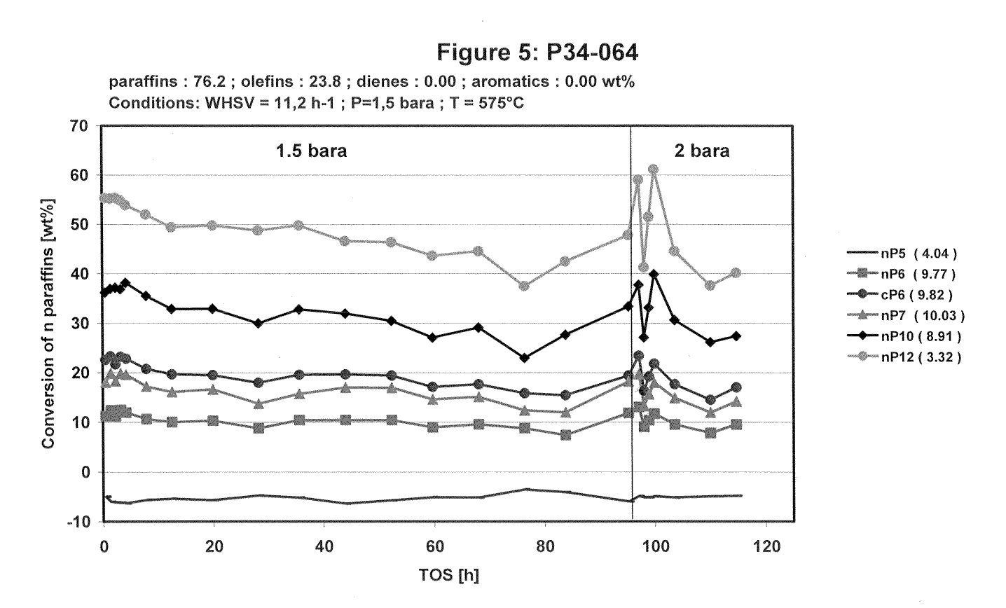

FIG. 5 shows the relationship between the conversion of the paraffins with respect to the time on stream (TOS) for Example 2 of the invention;

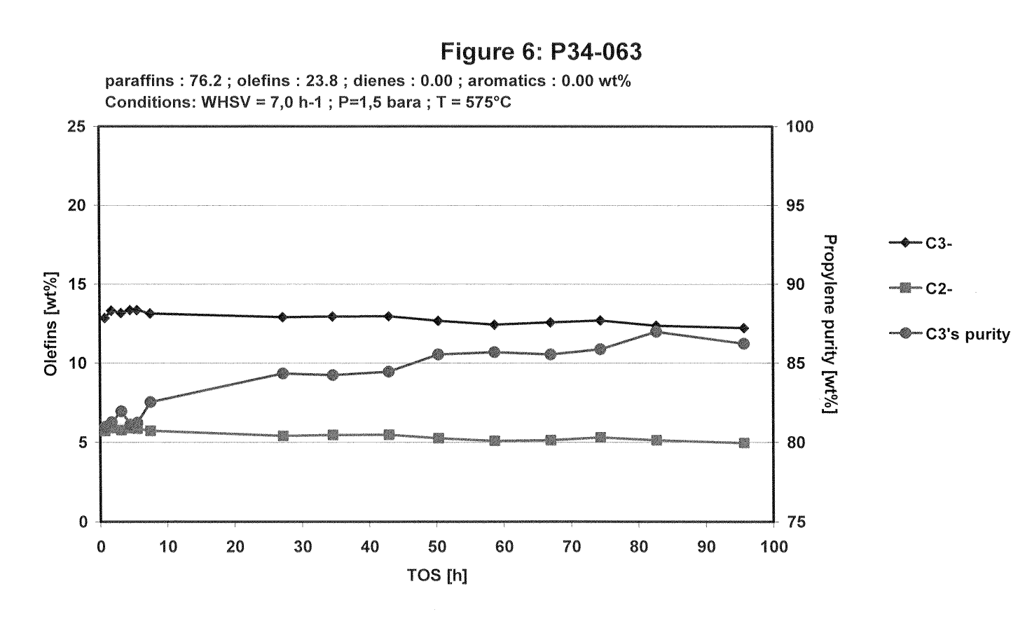

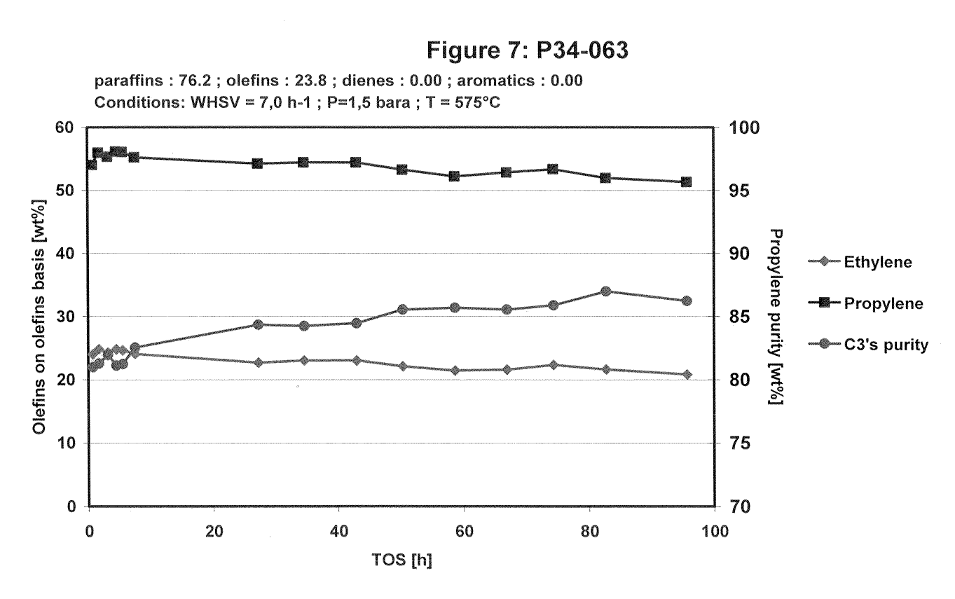

FIGS. 6 and 7 show the relationship between the olefins in the effluent and the propylene purity with respect to the time on stream (TOS) (FIG. 6) and the relationship between the olefin yield on an olefins basis and the time on stream (TOS) (FIG. 7) for Example 3 of the invention;

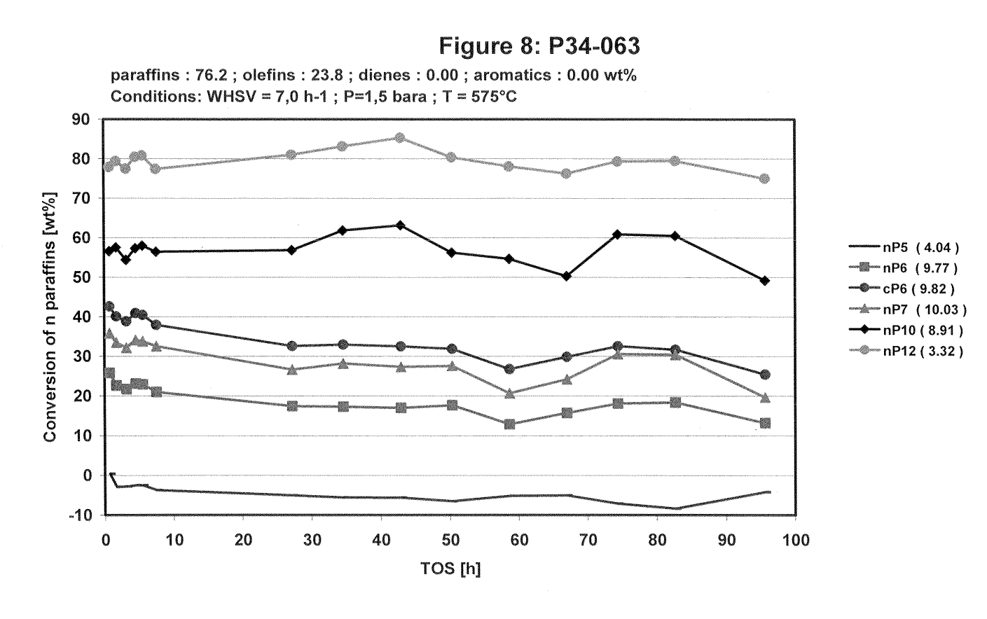

FIG. 8 shows the relationship between the conversion of the paraffins with respect to the time on stream (TOS) for Example 3 of the invention;

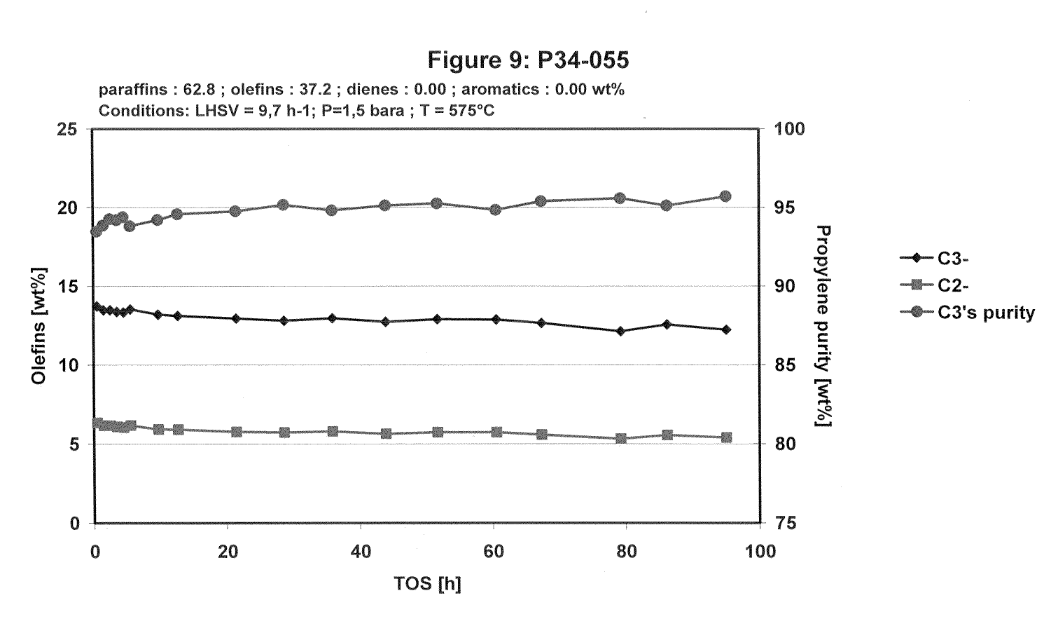

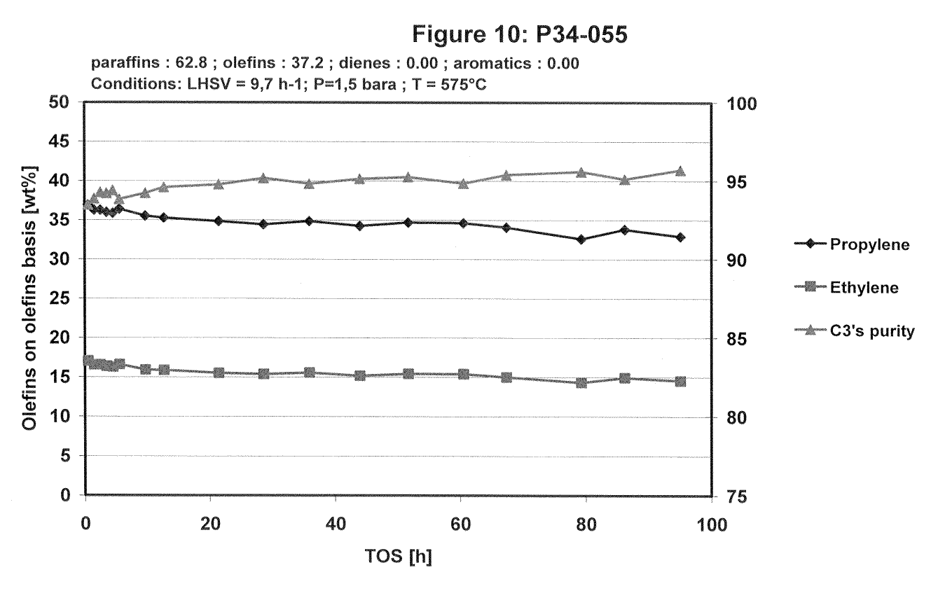

FIGS. 9 and 10 show the relationship between the olefins in the effluent and the propylene purity with respect to the time on stream (TOS) (FIG. 9) and the relationship between the olefin yield on an olefins basis and the time on stream (TOS) (FIG. 10) for Example 4 of the invention;

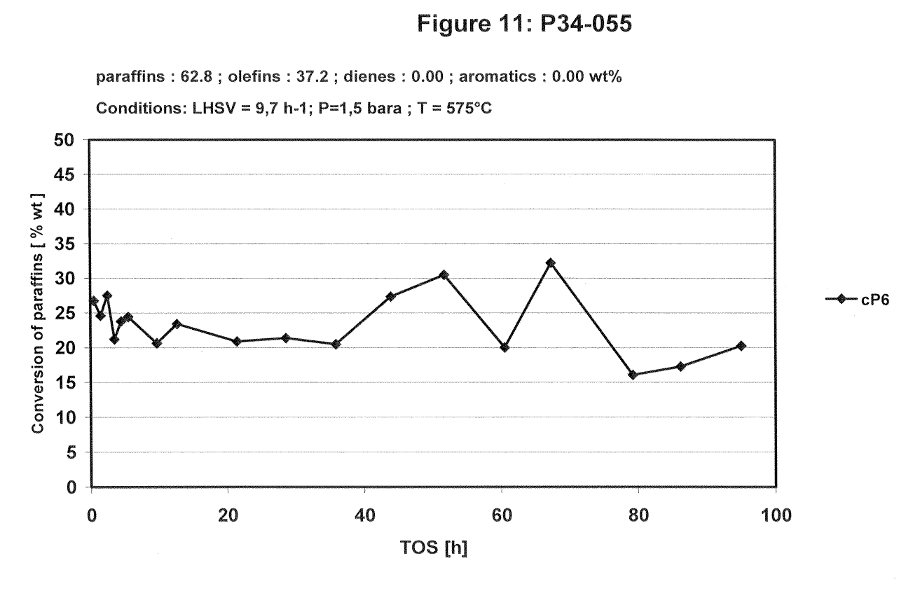

FIG. 11 shows the relationship between the conversion of the cy-C6 paraffin with respect to the time on stream (TOS) for Example 4 of the invention;

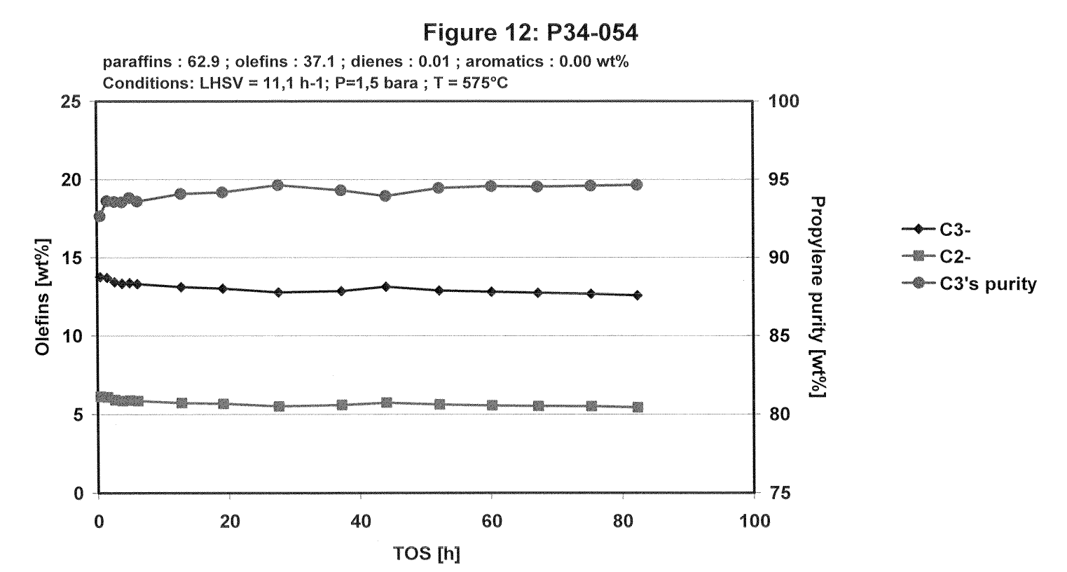

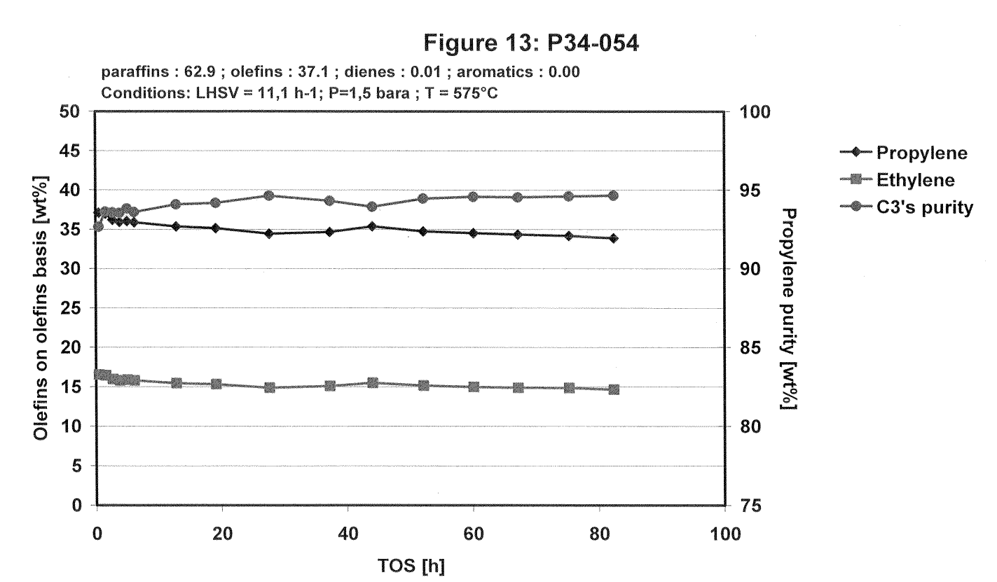

FIGS. 12 and 13 show the relationship between the olefins in the effluent and the propylene purity with respect to the time on stream (TOS) (FIG. 12) and the relationship between the olefin yield on an olefins basis and the time on stream (TOS) (FIG. 13) for Example 5 of the invention;

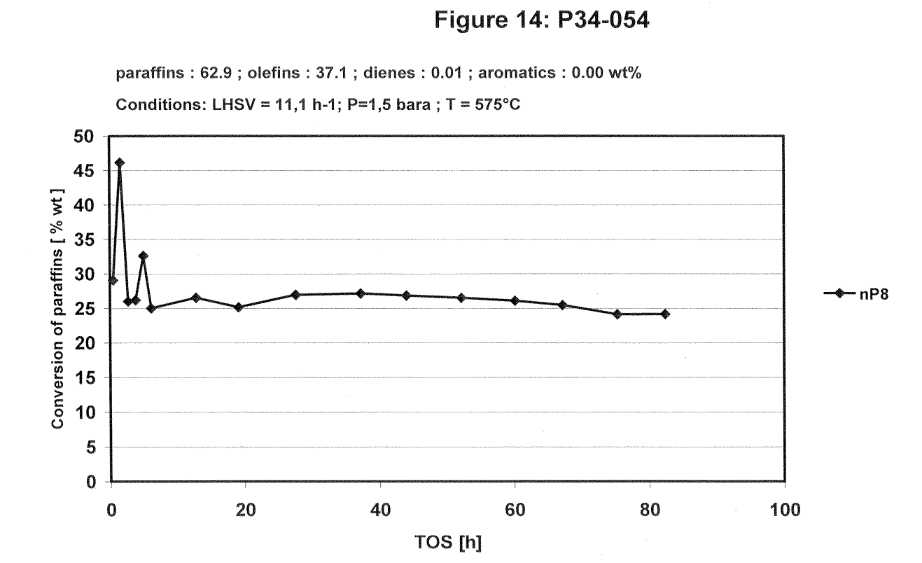

FIG. 14 shows the relationship between the conversion of the n-C8 paraffin with respect to the time on stream (TOS) for Example 5 of the invention;

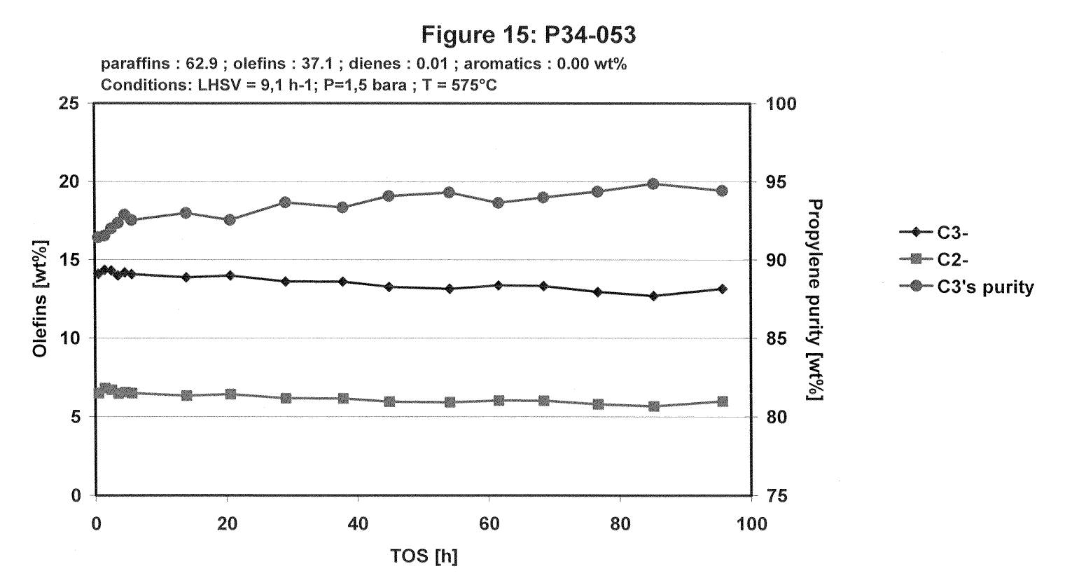

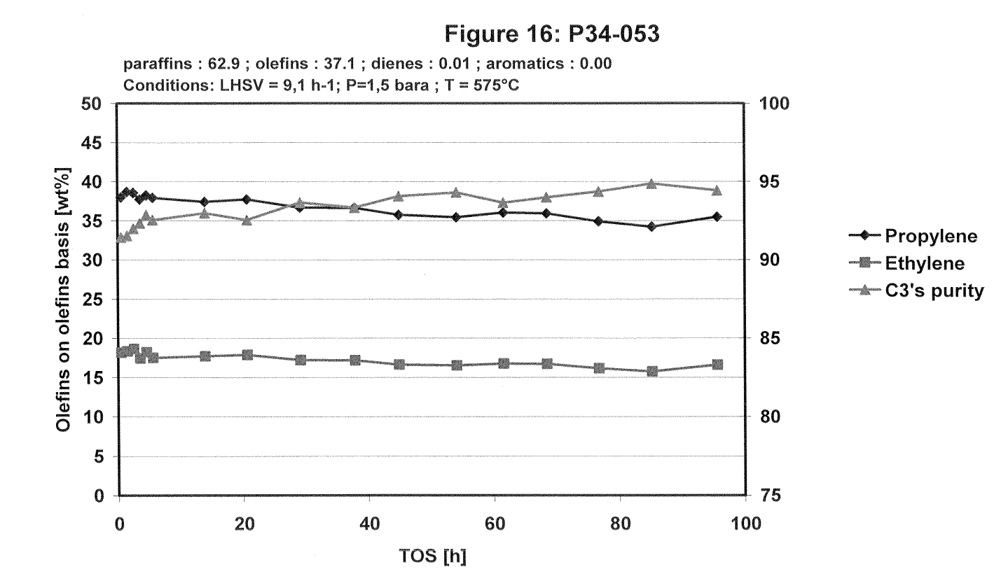

FIGS. 15 and 16 show the relationship between the olefins in the effluent and the propylene purity with respect to the time on stream (TOS) (FIG. 15) and the relationship between the olefin yield on an olefins basis and the time on stream (TOS) (FIG. 16) for Example 6 of the invention;

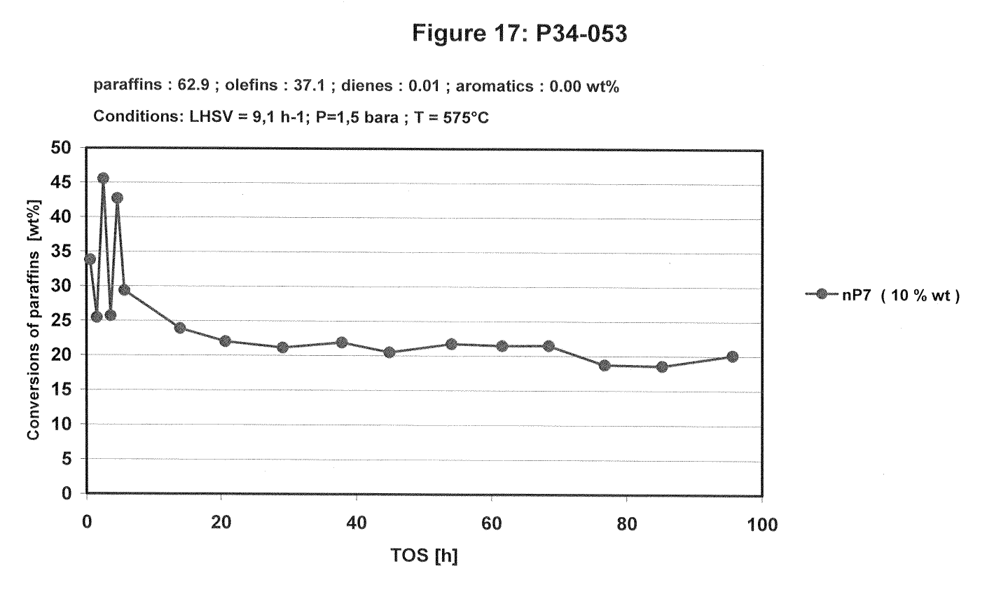

FIG. 17 shows the relationship between the conversion of the n-C7 paraffin with respect to the time on stream (TOS) for Example 6 of the invention;

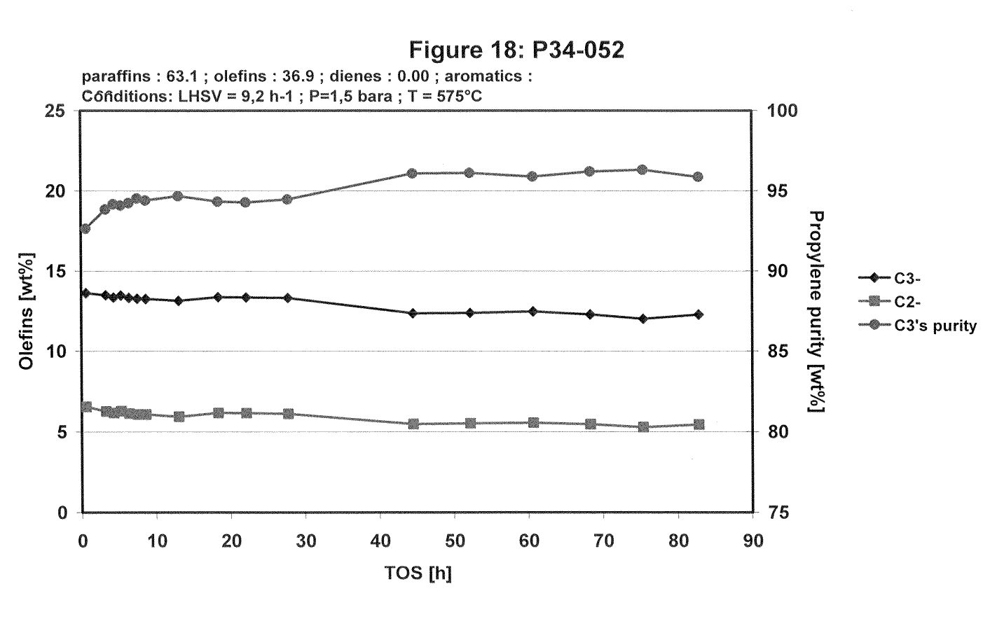

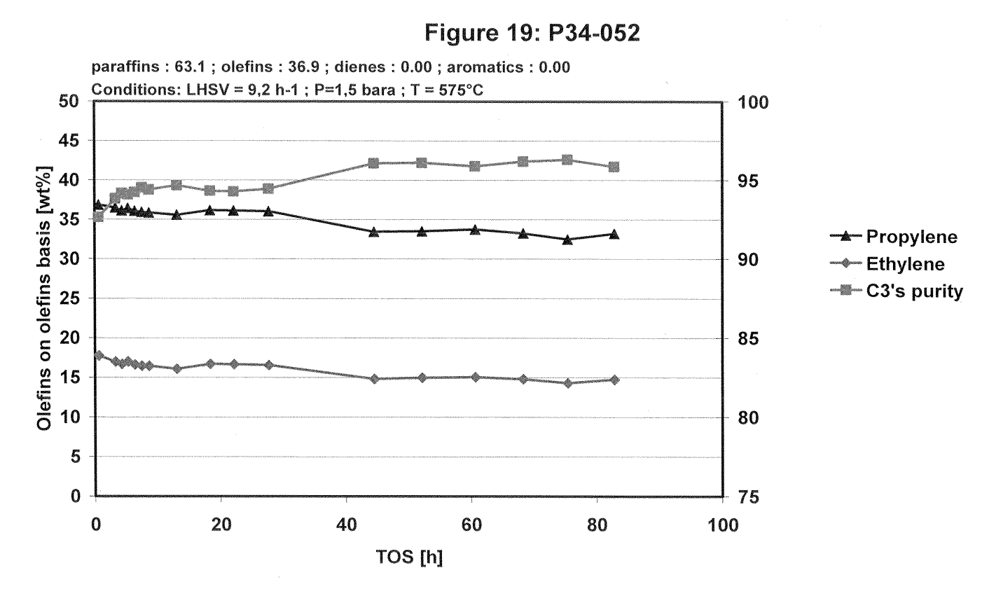

FIGS. 18 and 19 show the relationship between the olefins in the effluent and the propylene purity with respect to the time on stream (TOS) (FIG. 18) and the relationship between the olefin yield on an olefins basis and the time on stream (TOS) (FIG. 19) for Example 7 of the invention;

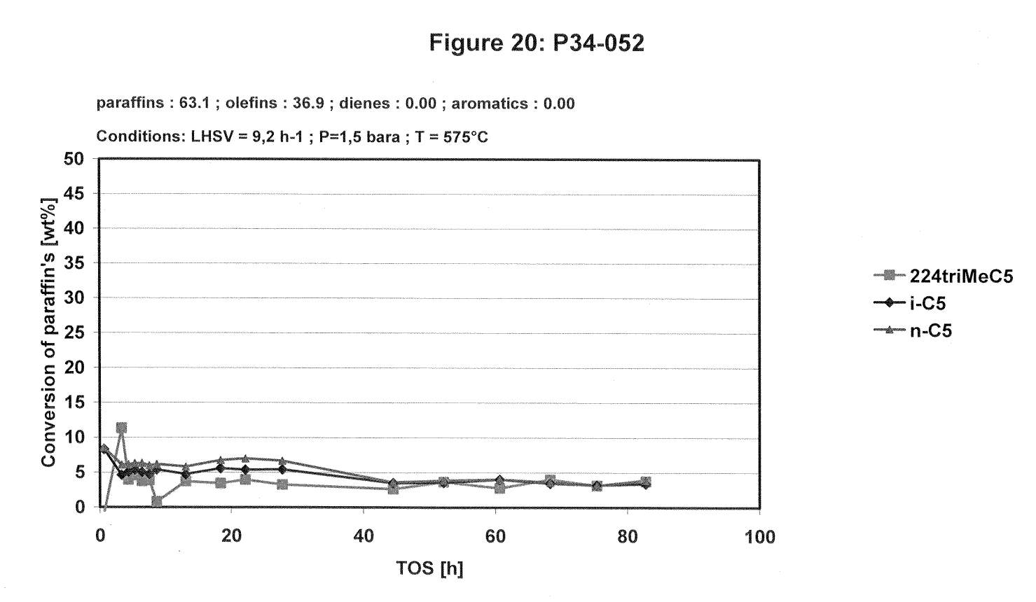

FIG. 20 shows the relationship between the conversion of the paraffins with respect to the time on stream (TOS) for Example 7 of the invention;

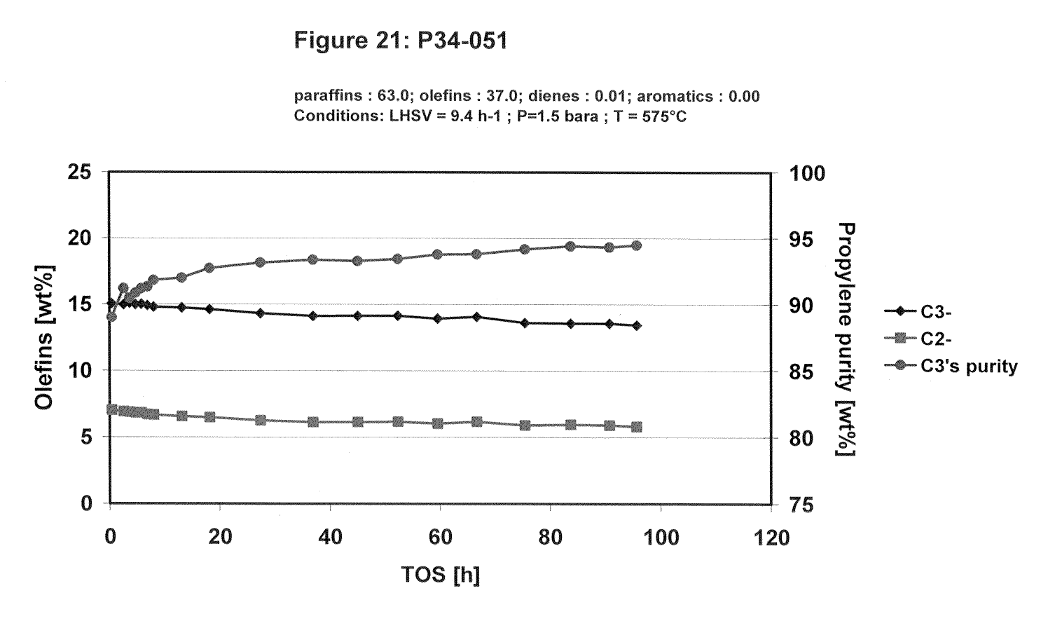

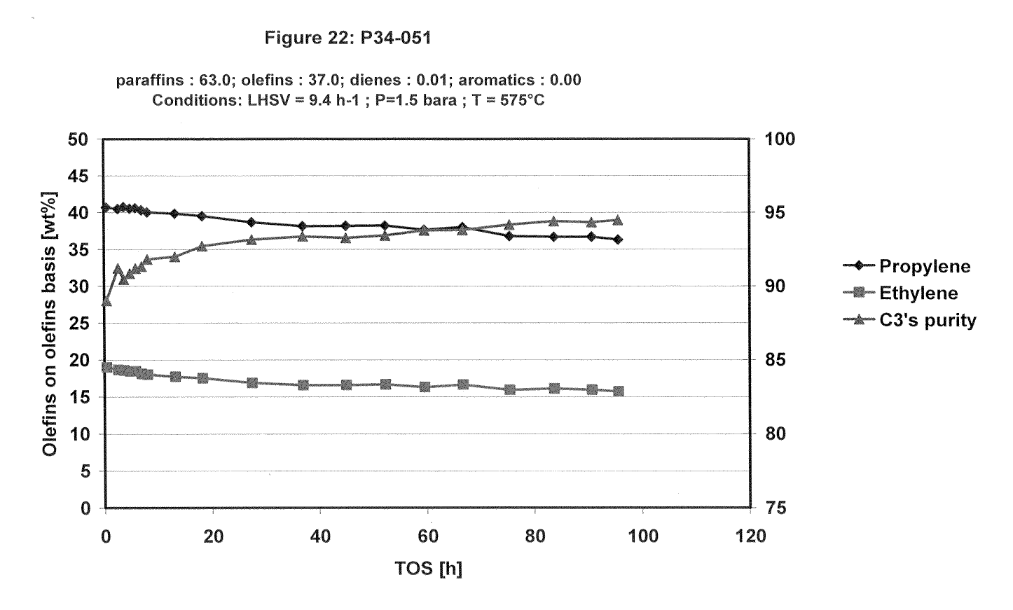

FIGS. 21 and 22 show the relationship between the olefins in the effluent and the propylene purity with respect to the time on stream (TOS) (FIG. 21) and the relationship between the olefin yield on an olefins basis and the time on stream (TOS) (FIG. 22) for Example 8 of the invention;

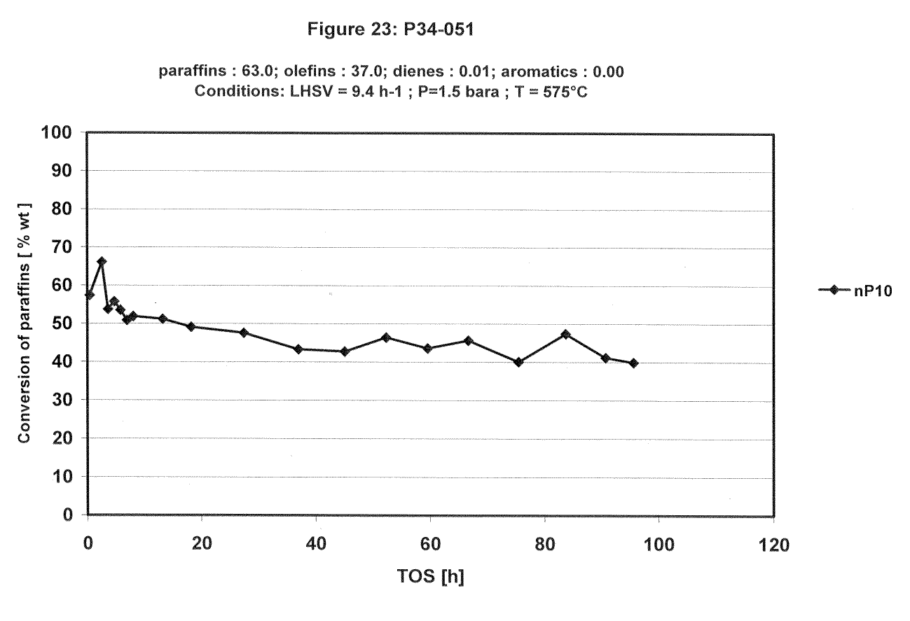

FIG. 23 shows the relationship between the conversion of the n-C10 paraffin with respect to the time on stream (TOS) for Example 8 of the invention;

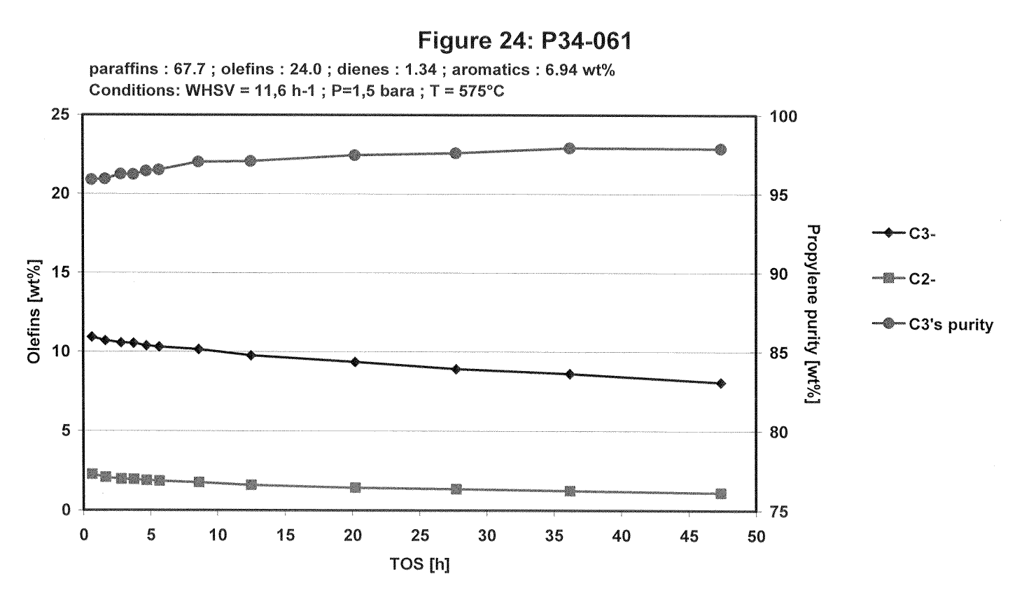

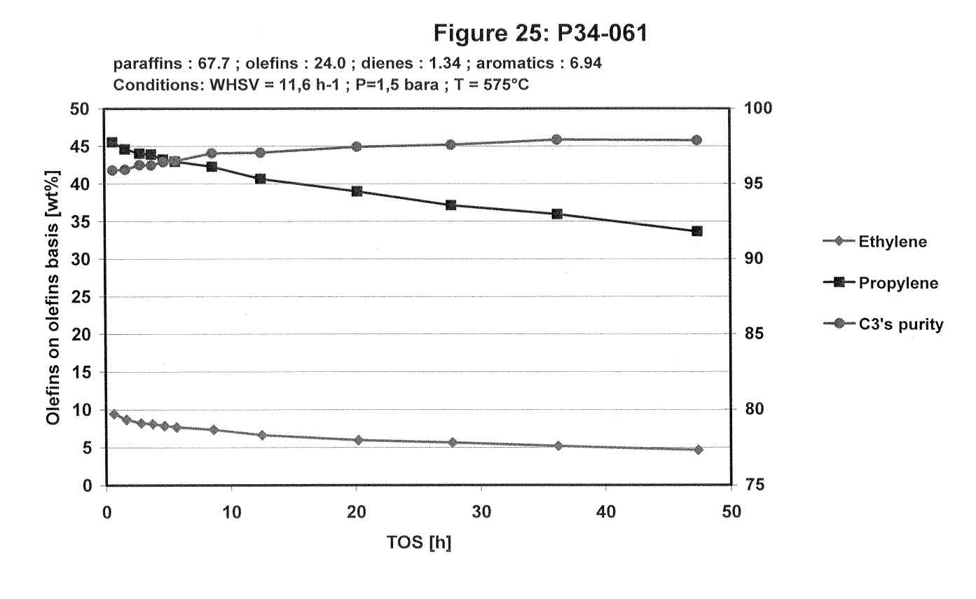

FIGS. 24 and 25 show the relationship between the olefins in the effluent and the propylene purity with respect to the time on stream (TOS) (FIG. 24) and the relationship between the olefin yield on an olefins basis and the time on stream (TOS) (FIG. 25) for Example 9 of the invention;

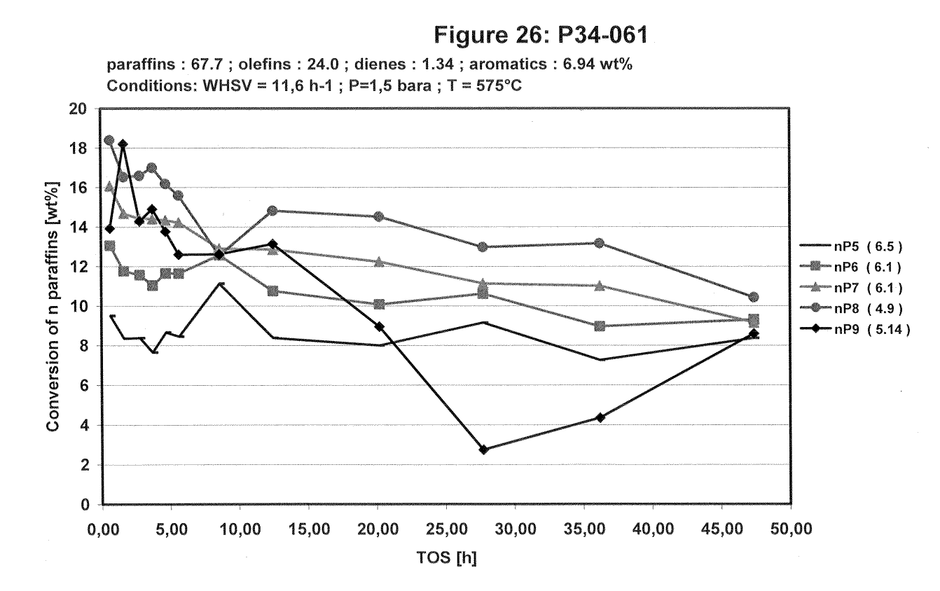

FIG. 26 shows the relationship between the conversion of the n-paraffins with respect to the time on stream (TOS) for Example 9 of the invention;

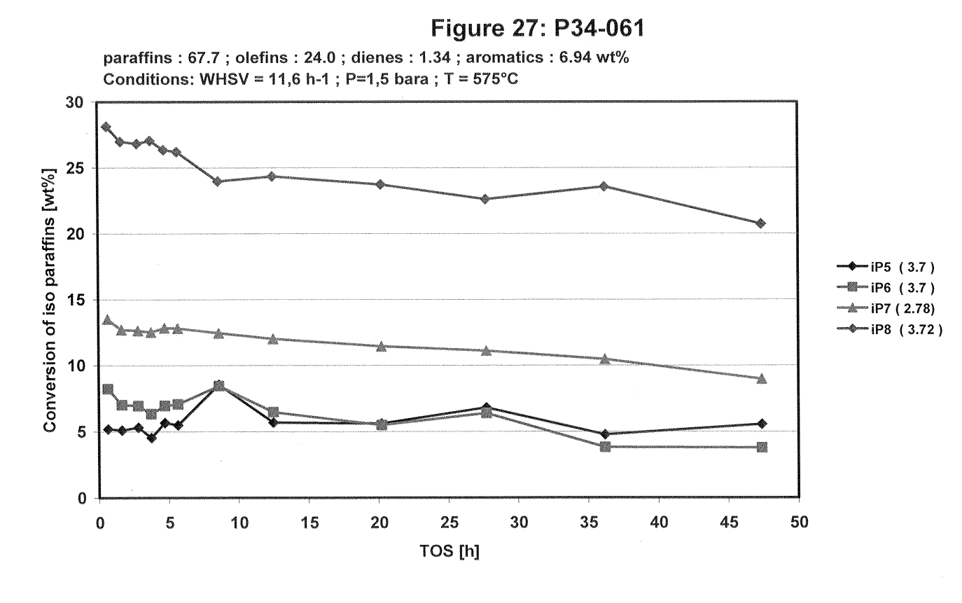

FIG. 27 shows the relationship between the conversion of the iso-paraffins with respect to the time on stream (TOS) for Example 9 of the invention; and

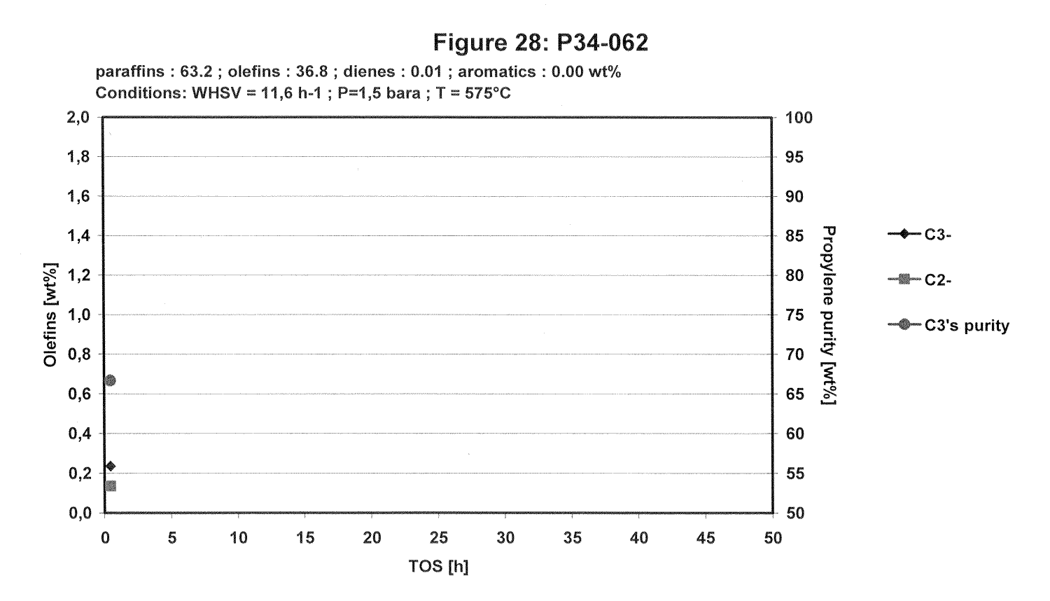

FIG. 28 shows the relationship between the olefins in the effluent and the propylene purity with respect to the time on stream (TOS) for Comparative Example 1.

In accordance with the present invention, catalytic conversion of a paraffin-containing feedstock into an effluent containing light olefins, in particular ethylene and propylene, and selectively into propylene is achieved. The process comprises passing a hydrocarbon feedstock containing at least 25 wt % C₅₊ paraffins through a reactor containing a crystalline silicate catalyst to produce an effluent including propylene. The paraffin containing feedstock may comprise a stream that has been derived from a refinery or petrochemical plant. Alternatively, it may have been formed by combining at least two such streams, optionally with a further stream of one or more paraffins. Therefore, the method may further comprise the step of forming the hydrocarbon feedstock by adding at least one C₄+ hydrocarbon feedstock cut comprising C₄₊ paraffins. The at least one C₆₊ paraffin may comprise a C₆₋₂₀ paraffin. The added paraffin may be linear. The hydrocarbon feedstock may comprise from 1 to 80 wt % of the at least one C₆₊ paraffin and from 20 to 99 wt % of the C₄+ hydrocarbon feedstock cut comprising C₄₊ paraffins.

The feedstock may comprise a single refinery stream including a mixture of paraffins including both linear paraffins, and iso-paraffins and cyclo-paraffins. An example is straight run naphtha, which comprises saturated C₅₋₉ paraffins and naphthenes. In the process of the invention, the linear paraffins are partially converted into olefins whereas the iso-paraffins and cyclo-paraffins are less converted or even substantially unconverted in case of multiple branched paraffins. This provides a process where the paraffinic content of the effluent is relatively richer in iso-paraffins than the feedstock, which effluent may be suitable for use as a feedstock for a subsequent steam cracking process or used as a blending feedstock for gasoline or kerosene production.

The hydrocarbon feedstock may contain at least one C₄₊ olefin in addition to the paraffins. These olefins are also converted into lower olefins such as propylene. This can additionally improve the thermal balance of the reactor, as compared to cracking of the paraffins alone.

In a specific embodiment the feedstock comprises 55 to 60 w % paraffins for respectively 45 to 40 w % olefins. In said paraffins there are 55 to 60 w % C5, in said olefins there are 20 to 30 w % C5.

In another specific embodiment the feedstock comprises 73 to 80 w % paraffins for respectively 27 to 20 w % olefins. In said paraffins there are C5 such as the C5 amount in the complete feedstock (paraffins+olefins) is at least 25%. In said olefins there are 12 to 18 w % C5.

In another specific embodiment the feedstock comprises 60 to 65 w % paraffins for respectively 40 to 35 w % olefins. In said paraffins there are 45 to 55 w % C5, in said olefins there are 20 to 35 w % C5.

In another specific embodiment the feedstock comprises, the total being 100 w %, 60 to 70% paraffins (comprising at least 42% C5), 20 to 30% olefins and 5 to 10% aromatics.

In accordance with the preferred process of the invention, the hydrocarbon feedstocks are selectively converted in the presence of an MFI-type or MEL-type crystalline silicate catalyst such as silicalite so as to produce propylene in the resultant effluent. The catalyst and process conditions are selected whereby the process has a particular yield towards propylene in the effluent.

In accordance with a preferred aspect of the present invention, the catalyst comprises a crystalline silicate of the MFI or MEL family, which may be a ZSM, a silicalite, or any other silicate in that family. The three-letter designation “MFI” or “MEL” represents a particular crystalline silicate structure type as established by the Structure Commission of the International Zeolite Association. Examples of MFI silicates are ZSM-5 and silicalite. Examples of MEL silicates are ZSM-11.

The preferred crystalline silicates have pores or channels defined by ten oxygen rings and a high silicon/aluminium atomic ratio.

Crystalline silicates are microporous crystalline inorganic polymers based on a framework of XO₄ tetrahedra linked to each other by sharing of oxygen ions, where X may be trivalent (e.g. Al, B, . . . ) or tetravalent (e.g. Ge, Si, . . . ). The crystal structure of a crystalline silicate is defined by the specific order in which a network of tetrahedral units are linked together. The size of the crystalline silicate pore openings is determined by the number of tetrahedral units, or, alternatively, oxygen atoms, required to form the pores and the nature of the cations that are present in the pores. They possess a unique combination of the following properties: high internal surface area; uniform pores with one or more discrete sizes; ion exchangeability; good thermal stability; and ability to adsorb organic compounds. Since the pores of these crystalline silicates are similar in size to many organic molecules of practical interest, they control the ingress and egress of reactants and products, resulting in particular selectivity in catalytic reactions. Crystalline silicates with the MFI structure possess a bi-directional intersecting pore system with the following pore diameters: a straight channel along [010]: 0.53-0.56 nm and a sinusoidal channel along [100]: 0.51-0.55 nm.

The crystalline silicate catalyst has structural and chemical properties and is employed under particular reaction conditions whereby the catalytic conversion to form light olefins, in particular propylene, readily proceeds.

The catalyst preferably has a high silicon/aluminium atomic ratio, whereby the catalyst has relatively low acidity. In this specification, the term “silicon/aluminium atomic ratio” is intended to mean the Si/Al atomic ratio of the overall material, which may be determined by chemical analysis. In particular, for crystalline silicate materials, the stated Si/Al ratios apply not just to the Si/Al framework of the crystalline silicate but rather to the whole material.

Different reaction pathways can occur on the catalyst. Hydrogen transfer reactions are directly related to the strength and density of the acid sites on the catalyst, and such reactions are preferably suppressed by the use of high Si/Al ratios so as to avoid the formation of coke during the conversion process, thereby increasing the stability of the catalyst. Moreover, the use of high Si/Al atomic ratios has been found to increase the propylene selectivity of the catalyst, i.e. to reduce the amount of propane produced and/or to increase the propylene/ethylene ratio. This increases the purity of the resultant propylene.

In accordance with one aspect, the crystalline silicate catalyst has a high silicon/aluminum atomic ratio of from 120 to 1000, more preferably from 180 to 500 whereby the catalyst has relatively low acidity. Hydrogen transfer reactions are directly related to the strength and density of the acid sites on the catalyst, and such reactions are preferably suppressed so as to avoid the progressive formation of coke, which in turn would otherwise decrease the stability of the catalyst over time. Such hydrogen transfer reactions tend to produce saturates such as intermediate unstable dienes and cyclo-olefins, and aromatics, none of which favours conversion into light olefins. Cyclo-olefins are precursors of aromatics and coke-like molecules, especially in the presence of solid acids, i.e. an acidic solid catalyst. The acidity of the catalyst can be determined by the amount of residual ammonia on the catalyst following contact of the catalyst with ammonia which adsorbs to the acid sites on the catalyst with subsequent ammonium desorption at elevated temperature measured by differential thermogravimetric analysis.

With such high silicon/aluminum ratio in the crystalline silicate catalyst, a stable conversion of the hydrocarbon feedstock can be achieved, with a high propylene yield of from 8 to 50%, more preferably from 12 to 35%. The propylene selectivity is such that in the effluent the propylene/ethylene weight ratio is typically from 2 to 5 and/or the propylene/propane weight ratio is typically from 5 to 30. Such high silicon/aluminum ratios in the catalyst reduce the acidity of the catalyst, thereby also increasing the stability of the catalyst.

The MFI or MEL catalyst having a high silicon/aluminum atomic ratio for use in the catalytic conversion process of the present invention may be manufactured by removing aluminum from a commercially available crystalline silicate: A typical commercially available silicalite has a silicon/aluminum atomic ratio of around 120. The commercially available MFI or MEL crystalline silicate may be modified by a steaming process which reduces the tetrahedral aluminum in the crystalline silicate framework and converts the aluminum atoms into octahedral aluminum in the form of amorphous alumina. Although in the steaming step aluminum atoms are chemically removed from the crystalline silicate framework structure to form alumina particles, those particles cause partial obstruction of the pores or channels in the framework. This inhibits the conversion processes of the present invention. Accordingly, following the steaming step, the crystalline silicate is subjected to an extraction step wherein amorphous alumina is removed from the pores and the micropore volume is, at least partially, recovered. The physical removal, by a leaching step, of the amorphous alumina from the pores by the formation of a water-soluble aluminum complex yields the overall effect of de-alumination of the MFI or MEL crystalline silicate. In this way by removing aluminum from the MFI or MEL crystalline silicate framework and then removing alumina formed therefrom from the pores, the process aims at achieving a substantially homogeneous de-alumination throughout the whole pore surfaces of the catalyst. This reduces the acidity of the catalyst, and thereby reduces the occurrence of hydrogen transfer reactions in the conversion process. The reduction of acidity ideally occurs substantially homogeneously throughout the pores defined in the crystalline silicate framework. This is because in the hydrocarbon conversion process hydrocarbon species can enter deeply into the pores. Accordingly, the reduction of acidity and thus the reduction in hydrogen transfer reactions which would improve the stability of the MFI or MEL catalyst are pursued throughout the whole pore structure in the framework. The framework silicon/aluminum ratio may be increased by this process to a value of from 150 to 500.

The MFI or MEL crystalline silicate catalyst may be mixed with a binder, preferably an inorganic binder, and shaped to a desired shape, e.g. extruded pellets. The binder is selected so as to be resistant to the temperature and other conditions employed in the catalyst manufacturing process and in the subsequent catalytic conversion process. The binder is an inorganic material selected from clays, silica, metal oxides such as ZrO₂ and/or metals, or gels including mixtures of silica and metal oxides. The binder is preferably alumina-free. However, aluminum in certain chemical compounds as in AlPO₄'s may be used, as the latter are quite inert and not acidic in nature. If the binder, which is used in conjunction with the crystalline silicate, is itself catalytically active, this may alter the conversion and/or the selectivity of the catalyst. Inactive materials for the binder may suitably serve as diluents to control the amount of conversion so that products can be obtained economically and orderly without employing other means for controlling the reaction rate. It is desirable to provide a catalyst having a good crush strength. This is because in commercial use, it is desirable to prevent the catalyst from breaking down into powder-like materials. Such clay or oxide binders have been employed normally only for the purpose of improving the crush strength of the catalyst. A particularly preferred binder for the catalyst of the present invention comprises silica.

The relative proportions of the finely divided crystalline silicate material and the inorganic oxide matrix of the binder can vary widely. Typically, the binder content ranges from 5 to 95% by weight, more typically from 20 to 50% by weight, based on the weight of the composite catalyst. Such a mixture of crystalline silicate and an inorganic oxide binder is referred to as a formulated crystalline silicate.

In mixing the catalyst with a binder, the catalyst may be formulated into pellets, extruded into other shapes, or formed into a spray-dried powder.

Typically, the binder and the crystalline silicate catalyst are mixed together by an extrusion process. In such a process, the binder, for example silica, in the form of a gel is mixed with the crystalline silicate catalyst material and the resultant mixture is extruded into the desired shape, for example pellets. Thereafter, the formulated crystalline silicate is calcined in air or an inert gas, typically at a temperature of from 200 to 900° C. for a period of from 1 to 48 hours.

The binder preferably does not contain any aluminium compounds, such as alumina. This is because as mentioned above the preferred catalyst has a selected silicon/aluminium ratio of the crystalline silicate. The presence of alumina in the binder yields other excess alumina if the binding step is performed prior to the aluminium extraction step. If the aluminium-containing binder is mixed with the crystalline silicate catalyst following aluminium extraction, this re-aluminates the catalyst. The presence of aluminium in the binder would tend to reduce the propylene selectivity of the catalyst, and to reduce the stability of the catalyst over time.

In addition, the mixing of the catalyst with the binder may be carried out either before or after any optional steaming step.

The various preferred catalysts have been found to exhibit high stability, in particular being capable of giving a stable propylene yield over several days, e.g. up to five days. This enables the catalytic conversion process to be performed continuously in two parallel “swing” reactors wherein when one reactor is operating, the other reactor is undergoing catalyst regeneration. The catalyst also can be regenerated several times. The catalyst is also flexible in that it can be employed to crack a variety of feedstocks, either pure or mixtures, coming from different sources in the oil refinery or petrochemical plant and having different compositions.

In the catalytic conversion process, the process conditions are selected in order to provide high selectivity towards propylene, a stable conversion into propylene over time, and a stable product distribution in the effluent. Such objectives are favoured by the use of a low acid density in the catalyst (i.e. a high Si/Al atomic ratio) in conjunction with a low pressure, a high inlet temperature and a short contact time, all of which process parameters are interrelated and provide an overall cumulative effect (e.g. a higher pressure may be offset or compensated by a yet higher inlet temperature). The process conditions are selected to disfavour hydrogen transfer reactions leading to the formation of aromatics and coke precursors. The process operating conditions thus employ a high space velocity, a low pressure and a high reaction temperature.

The liquid hourly space velocity (LHSV) with respect to the hydrocarbon feedstock preferably ranges from 5 to 30 h⁻¹, more preferably from 5 to 15 h⁻¹. The paraffin-containing hydrocarbon feedstock is preferably fed at a total inlet pressure sufficient to convey the feedstock through the reactor. Preferably, the total absolute pressure in the reactor ranges from 0 to 2 bars. Preferably, the inlet temperature of the feedstock ranges from 500 to 600° C., more preferably from 550 to 600° C., yet more preferably about 575° C.

The catalytic conversion process can be performed in a fixed bed reactor, a moving bed reactor or a fluidized bed reactor. A typical fluid bed reactor is one of the FCC type used for fluidized-bed catalytic cracking in the oil refinery. A typical moving bed reactor is of the continuous catalytic reforming type. As described above, the process may be performed continuously using a pair of parallel “swing” fixed bed reactors.

Since the catalyst exhibits high stability for an extended period, typically at least around five days, the frequency of regeneration of the catalyst is low. More particularly, the catalyst may accordingly have a lifetime which exceeds one year.

The light fractions of the effluent, namely the C₂ and C₃ cuts, can contain more than 90% olefins (i.e. ethylene and propylene). Such cuts are sufficiently pure to constitute chemical grade olefin feedstocks. The propylene yield in such a process can range from 8 to 50%. The propylene/ethylene weight ratio typically ranges from 2 to 5, more typically from 2.5 to 4.0. The propylene/propane weight ratio typically ranges from 5 to 30, more typically from 8 to 20. These ratios may be higher than obtainable using the known thermal cracking process for producing olefins from paraffins described herein.

The present invention will now be described in greater detail with reference to the following non-limiting Examples.

### EXAMPLE 1

### P34-057

In Example 1, a laboratory scale fixed bed reactor had provided therein a formulated crystalline silicate catalyst of the MFI-type. The catalyst comprises silicalite, which had a silicon/aluminium atomic ratio of 268 (0.168 wt % of aluminium).

The catalyst was a silicalite catalyst available from UOP (14/7499; UOP#62-1770) and was in the form of trilobes. The formulated catalyst was crushed and the particles of 35 to 45 mesh size retained for the test.

The laboratory scale reactor had a diameter of 11 mm and was loaded with a catalyst load of about 6.7 g. The reactor was operated at a pressure of 1.5 bara at the outlet. The reactor was fed with a hydrocarbon feedstock, which was a C₅ gasoline base cut. The combined feedstock contained approximately 58.9 wt % paraffins and 41.1 wt % olefins and had the following primary paraffinic components (in approximate weight percent): i-C5 50.71 wt % and n-C5 6.93 wt % and having the following primary olefinic components (in approximate weight percent): i-C5-2.73 wt %, t-2C5-16.19 wt %, c-2c5-7.25 wt %, 2Me2C4-7.40 wt %, and cy-C5-2.68 wt %. The LHSV was 9.45 h⁻¹. The reactor inlet temperature was 575° C. The composition of the effluent was analysed over a period of time. The relationship between the olefins in the effluent and the olefins purity with respect to the time on stream (TOS) is shown in FIG. 1 and the relationship between the olefin yield on an olefins basis and the time on stream (TOS) is shown in FIG. 2.

The yield on olefin basis is defined as the yield on feed basis divided by the olefin content of the feed.

It may be seen from FIG. 1, the propylene comprised about 14-13 wt % of the effluent and the C₃ purity was greater than 95% of propylene. FIG. 2 shows that the propylene yield on an olefins basis was above 30%. The propylene yield was consistently maintained over a TOS of nearly 100 hours.

The initial olefin/paraffin weight ratio of the feedstock was 0.70 whereas the final olefin/paraffin weight ratio of the effluent was 0.65. Therefore the proportion of olefins as a whole was decreased as a result of the catalytic cracking process, even though significant propylene was produced in the effluent.

At the end of the catalytic test, the catalyst was regenerated using 2 vol % of oxygen in nitrogen at a temperature starting from 530° C. and ending at 575° C. over a time of about 24 hours. The reactor was purged with nitrogen before introducing hydrocarbon feed.

### EXAMPLE 2

### P34-064

In Example 2 the process of Example 1 was repeated with the same catalyst, pressure and reactor inlet temperature. The LHSV was slightly increased. The feedstock was modified by the addition of additional paraffinic components to the gasoline cut of Example 1. The results are shown in FIGS. 3, 4 and 5.

The reactor was fed with a hydrocarbon feedstock, which was a C₅ gasoline base cut (used in Example 1) to which had been added additional cyclo- and n-paraffins (cy-C6, n-C6, n-C7, n-C10 and n-C12). The combined feedstock contained approximately 76.2 wt % paraffins and 23.8 wt % olefins and had the following primary paraffinic components (in approximate weight percent): i-C5 29.36 wt %, n-C5 4.04 wt %, n-C6 9.78 wt %, cy-C6 9.83 wt %, n-C₇-10.04 wt %, n-C10 8.92 wt % and n-C12 3.33 wt % and having the following primary olefinic components (in approximate weight percent): i-C5-1.57 wt %, t-2C5-9.44 wt %, c-2c5-4.14 wt %, 2Me2C4-4.33 wt %, and cy-C5-1.59 wt %. The LHSV was 11.2 h⁻¹. The reactor inlet temperature was 575° C. The composition of the effluent was analysed over a period of time.

After approximately 95 hours on stream, the pressure was increased to 2 bara.

From the FIGS. 3 and 4 it may be seen that the yield of propylene was about 11-10 wt % giving a propylene yield on an olefins basis of above 40 wt %. The propylene purity was about 90%. Increasing the pressure tended to reduce these values after an initial stabilisation period.

FIG. 5 shows that the C6+ paraffins were converted during the process and also that the degree of conversion tended generally to reduce over time. This conversion indicated that the paraffin molecules, particularly the linear higher carbon C6 and above paraffins, were partially catalytically cracked into lower olefins. The highest conversion was for the n-C12 paraffin which ranged from about 50 to about 40% over the TOS. There were progressively lower conversions for the n-P10, c-P6, n-P7 and n-P6 paraffins. There was a negative conversion for the n-P5 paraffin. Therefore the higher carbon (nP7, nP10 and nP12) linear paraffins were more converted than the lower carbon (n-P6 and n-P5) linear paraffins. The cyclo paraffin c-P6 was also converted, at a proportion greater than for the corresponding linear paraffin n-P6 These effects were surprising, and indicate that the addition of linear paraffins, particularly C6+ paraffins, or cyclo-paraffins would lead to higher propylene yields and higher conversion of paraffins into useful lower olefins.

FIG. 5 also shows that increasing the pressure can slightly increase the conversion of the paraffins.

The initial olefin/paraffin weight ratio of the feedstock was 0.31 whereas the final olefin/paraffin weight ratio of the effluent was 0.43. Therefore the proportion of olefins as a whole was increased as a result of the catalytic cracking process, and significant propylene was produced in the effluent.

At the end of the catalytic test, the catalyst was regenerated using 2 vol % of oxygen in nitrogen at a temperature starting from 530° C. and ending at 575° C. over a time of about 24 hours. The reactor was purged with nitrogen before introducing hydrocarbon feed.

### EXAMPLE 3

### P34-063

In Example 3 the process of Example 2 was repeated with the same catalyst, pressure and reactor inlet temperature, and the same feedstock. The LHSV was reduced to 7 h⁻¹. The results are shown in FIGS. 6, 7 and 8.

From the FIGS. 6 and 7 it may be seen that the yield of propylene was about 13-12 wt % giving a propylene yield on an olefins basis of above 50 wt %. The propylene purity was about 85%. Therefore, as compared to Example 2, decreasing the LHSV tended to increase the propylene yield but decrease its purity.

FIG. 8 shows that, like FIG. 5, conversion of the paraffins for C6+ paraffins, and also that lowering the LHSV (as compared to Example 2) tended to increase the paraffin conversion in the catalytic cracking process.

The initial olefin/paraffin weight ratio of the feedstock was 0.31 whereas the final olefin/paraffin weight ratio of the effluent was 0.49. Therefore the proportion of olefins as a whole was increased as a result of the catalytic cracking process, and significant propylene was produced in the effluent.

At the end of the catalytic test, the catalyst was regenerated using 2 vol % of oxygen in nitrogen at a temperature starting from 530° C. and ending at 575° C. over a time of about 24 hours. The reactor was purged with nitrogen before introducing hydrocarbon feed.

### EXAMPLE 4

### P34-055

In Example 4 the process of Example 1 was repeated with the same catalyst, pressure and reactor inlet temperature. The LHSV was slightly increased. The feedstock was modified by the addition of an additional cyclo-paraffinic component in the form of approximately 10 wt % cy-C6 to the gasoline cut of Example 1. The results are shown in FIGS. 9, 10 and 11.

The combined feedstock contained approximately 62.8 wt % paraffins and 37.2 wt % olefins and had the following primary paraffinic components (in approximate weight percent): i-C5 45.56 wt %, n-C5 6.29 wt %, and cy-C6 9.83 wt %, and having the following primary olefinic components (in approximate weight percent): i-C5-2.45 wt %, t-2C5-14.67 wt %, c-2c5-6.57 wt %, 2Me2C4-6.73 wt %, and cy-C5-2.47 wt %. The LHSV was 9.7 h⁻¹. The reactor inlet temperature was 575° C. The composition of the effluent was analysed over a period of time.

From the FIGS. 9 and 10 it may be seen that the yield of propylene was about 13-12 wt % giving a propylene yield on an olefins basis of about 35 wt %. The propylene purity was about 95%.

FIG. 11 shows that the added cP6 paraffin was converted at a proportion of about 20 to 25% and this tended slightly to reduce over time. This indicated that the cP6 paraffin molecules were partially catalytically cracked into lower olefins.

The initial olefin/paraffin weight ratio of the feedstock was 0.59 and the final olefin/paraffin weight ratio of the effluent was 0.59. Therefore the proportion of olefins as a whole was increased as a result of the catalytic cracking process as compared to Example 1.

At the end of the catalytic test, the catalyst was regenerated using 2 vol % of oxygen in nitrogen at a temperature starting from 530° C. and ending at 575° C. over a time of about 24 hours. The reactor was purged with nitrogen before introducing hydrocarbon feed.

### EXAMPLE 5

### P34-054

In Example 5 the process of Example 1 was repeated with the same catalyst, pressure and reactor inlet temperature. The LHSV was slightly increased. The feedstock was modified by the addition of an additional linear paraffinic component in the form of approximately 10 wt % n-C8 to the gasoline cut of Example 1. The results are shown in FIGS. 12, 13, 14 and 15.

The combined feedstock contained approximately 62.9 wt % paraffins and 37.1 wt % olefins and had the following primary paraffinic components (in approximate weight percent): i-C5 45.51 wt %, n-C5 6.27 wt %, and n-C8 9.87 wt %, and having the following primary olefinic components (in approximate weight percent): i-C5-2.45 wt %, t-2C5-14.64 wt %, c-2c5-6.56 wt %, 2Me2C4-6.73 wt %, and cy-C5-2.46 wt %. The LHSV was 11.1 h⁻¹. The reactor inlet temperature was 575° C. The composition of the effluent was analysed over a period of time.

From the FIGS. 12 and 13 it may be seen that the yield of propylene was about 13-12 wt % giving a propylene yield on an olefins basis of about 35 wt %. The propylene purity was about 95%.

FIG. 14 shows that the added nP8 paraffin was converted at a proportion of about 25% and this was generally constant over time. This indicated that the nC8 paraffin molecules were partially catalytically cracked into lower olefins.

The initial olefin/paraffin weight ratio of the feedstock was 0.59 and the final olefin/paraffin weight ratio of the effluent was 0.58. Therefore the proportion of olefins as a whole was slightly decreased as a result of the catalytic cracking process, and increased as compared to Example 1, and significant propylene was produced in the effluent.

At the end of the catalytic test, the catalyst was regenerated using 2 vol % of oxygen in nitrogen at a temperature starting from 530° C. and ending at 575° C. over a time of about 24 hours. The reactor was purged with nitrogen before introducing hydrocarbon feed.

### EXAMPLE 6

### P34-053

In Example 6 the process of Example 1 was repeated with the same catalyst, pressure and reactor inlet temperature. The LHSV was slightly reduced. The feedstock was modified by the addition of an additional paraffinic component in the form of approximately 10 wt % n-C7 to the gasoline cut of Example 1. The results are shown in FIGS. 15, 16 and 17.

The combined feedstock contained approximately 62.9 wt % paraffins and 37.1 wt % olefins and had the following primary paraffinic components (in approximate weight percent): i-C5 45.54 wt %, n-C5 6.28 wt %, and n-C7 9.86 wt %, and having the following primary olefinic components (in approximate weight percent): i-C5-2.45 wt %, t-2C5-14.65 wt %, c-2c5-6.56 wt %, 2Me2C4-6.73 wt %, and cy-C5-2.47 wt %. The LHSV was 9.1 h⁻¹. The reactor inlet temperature was 575° C. The composition of the effluent was analysed over a period of time.

From the FIGS. 15 and 16 it may be seen that the yield of propylene was about 14-13 wt % giving a propylene yield on an olefins basis of about 35 wt %. The propylene purity was about 93%.

FIG. 17 shows that the added n-C7 paraffin was converted at a proportion of about 20% which was generally constant over time. This indicated that the n-C7 paraffin molecules were partially catalytically cracked into lower olefins.

The initial olefin/paraffin weight ratio of the feedstock was 0.59 and the final olefin/paraffin weight ratio of the effluent was 0.58. Therefore the proportion of olefins as a whole was only slightly decreased as a result of the catalytic cracking process, as compared to Example 1, indicating that the linear-paraffin addition was significantly converted into propylene in the effluent.

At the end of the catalytic test, the catalyst was regenerated using 2 vol % of oxygen in nitrogen at a temperature starting from 530° C. and ending at 575° C. over a time of about 24 hours. The reactor was purged with nitrogen before introducing hydrocarbon feed.

### EXAMPLE 7

### P34-052

In Example 7 the process of Example 1 was repeated with the same catalyst, pressure and reactor inlet temperature. The LHSV was slightly reduced. The feedstock was modified by the addition of an additional paraffinic component in the form of approximately 10 wt % i-C8 (2,2,4-trimethylpentane) to the gasoline cut of Example 1. The results are shown in FIGS. 18, 19 and 20.

The combined feedstock contained approximately 63.1 wt % paraffins and 36.9 wt % olefins and had the following primary paraffinic components (in approximate weight percent): i-C5 45.20 wt %, n-C5 6.27 wt %, and i-C8 10.44 wt %, and having the following primary olefinic components (in approximate weight percent): i-C5 9.11 wt %, n-O5 23.61 wt % and c-O5 2.46 wt %. The LHSV was 9.2 h⁻¹. The reactor inlet temperature was 575° C. The composition of the effluent was analysed over a period of time.

From the FIGS. 18 and 19 it may be seen that the yield of propylene was about 13-12 wt % giving a propylene yield on an olefins basis of about 34 to 35 wt %. The propylene purity was about 94 to 96%.

FIG. 20 shows that the conversion of the added iC8 (2,2,4 triMeC5) paraffin was low, about 3%, as was the conversion of the two C5 paraffins, n-C5 and i-C5. This indicated that the iC8 paraffin molecules were not significantly catalytically cracked into lower olefins as compared to the linear n-P8 paraffin of Example 5 (see FIG. 14). FIG. 20 shows that the corresponding conversions for the i-C5 and n-C5 were low and that these paraffin molecules were not significantly catalytically cracked into lower olefins.

The initial olefin/paraffin weight ratio of the feedstock was 0.58 and the final olefin/paraffin weight ratio of the effluent was 0.54. Therefore the proportion of olefins as a whole was decreased as a result of the catalytic cracking process, and similar to the corresponding result of Example 1, indicating that the iso-paraffin addition was not significantly converted into propylene in the effluent.

At the end of the catalytic test, the catalyst was regenerated using 2 vol % of oxygen in nitrogen at a temperature starting from 530° C. and ending at 575° C. over a time of about 24 hours. The reactor was purged with nitrogen before introducing hydrocarbon feed.

### EXAMPLE 8

### P34-051

In Example 8 the process of Example 1 was repeated with the same catalyst, pressure and reactor inlet temperature. The LHSV was slightly reduced. The feedstock was modified by the addition of an additional paraffinic component in the form of approximately 10 wt % n-C10 to the gasoline cut of Example 1. The results are shown in FIGS. 21, 22 and 23.

The combined feedstock contained approximately 63.0 wt % paraffins and 37.0 wt % olefins and had the following primary paraffinic components (in approximate weight percent): i-C5 45.30 wt %, n-C5 6.26 wt %, and n-C10 10.19 wt %, and having the following primary olefinic components (in approximate weight percent): i-C5 9.12 wt %, n-O5 23.59 wt % and c-O5 2.48 wt %. The LHSV was 9.4 h⁻¹. The reactor inlet temperature was 575° C. The composition of the effluent was analysed over a period of time.

From the FIGS. 21 and 22 it may be seen that the yield of propylene was about 15-14 wt % giving a propylene yield on an olefins basis of about 37 wt %. The propylene purity was about 93%.

FIG. 23 shows that the conversion of the linear n-C10 molecule was high, generally above 40%, and that this conversion of the added n-C10 paraffin tended generally to reduce over time. This indicated that the n-C10 paraffin molecules were partially catalytically cracked into lower olefins.

The initial olefin/paraffin weight ratio of the feedstock was 0.59 and the final olefin/paraffin weight ratio of the effluent was 0.62. Therefore the proportion of olefins as a whole was increased as a result of the catalytic cracking process indicating that the linear-paraffin addition was significantly converted into propylene in the effluent.

At the end of the catalytic test, the catalyst was regenerated using 2 vol % of oxygen in nitrogen at a temperature starting from 530° C. and ending at 575° C. over a time of about 24 hours. The reactor was purged with nitrogen before introducing hydrocarbon feed.

### EXAMPLE 9

### P34-061

In Example 9 the process of Example 1 was repeated with the same catalyst, pressure and reactor inlet temperature. The feedstock was a coker naphtha. The results are shown in FIGS. 24, 256, 26 and 27.

The feedstock contained approximately 67.7 wt % paraffins, 24.0 wt % olefins, 1.34 wt % dienes, and 6.94 wt % aromatics. The WHSV was 11.6 h⁻¹ or 76.9 gr/h of feed was sent over 6.64 gr of catalyst. The reactor inlet temperature was 575° C. The composition of the effluent was analysed over a period of time.

From the FIGS. 24 and 25 it may be seen that the yield of propylene was less than about 10 wt % giving a propylene yield on an olefins basis of above 35 wt %. These values both decreased over the time on stream (TOS). The propylene purity was above 95%, and increased over the time on stream (TOS).

FIG. 26 shows the proportions of the linear nC5 to nC9 paraffins that were converted in the catalytic cracking process. The n-C8 paraffin had the highest conversion, at about 13 to 15%, with progressively lower conversion proportions with lower carbon number. The conversion of the n-P5 to n-P8 paraffins tended generally to reduce over time. This indicated that these linear paraffin molecules, particularly for higher carbon number, were partially catalytically cracked into lower olefins.

FIG. 27 shows that the proportions of the iC5 to iC8 paraffins that were converted in the catalytic cracking process. The i-C8 paraffin had the highest conversion, at about 23%, with progressively lower conversion proportions with lower carbon number. This indicated that these non-linear paraffin molecules were partially catalytically cracked into lower olefins Compared to example 7, where 2,2,4-trimethylpentane was added to the hydrocarbon feed, in the present example the i-C8 are mainly mono-methyl-heptanes. This example shows that mono-methyl-branched paraffins can still easily be cracked whereas multiple-branched paraffins can not.

The initial olefin/paraffin weight ratio of the feedstock was 0.35 and the final olefin/paraffin weight ratio of the effluent was 0.56. Therefore the proportion of olefins as a whole was increased as a result of the catalytic cracking process, indicating that significant propylene was produced in the effluent.

### COMPARATIVE EXAMPLE 1

In this Comparative Example, Example 8 was repeated without loading a catalyst in the same reactor tube. This was done to determine the degree of thermal cracking in the reactor (as compared to catalytic cracking). The feedstock was substantially the same as for Example 8 (the addition of an additional paraffinic component in the form of approximately 10 wt % n-C10 to the gasoline cut of Example 1 and contained approximately 63.2 wt % paraffins and 36.8 wt % olefins) and was fed thought the reactor at an WHSV of 11.6 as if there was catalyst in the reactor. This corresponds to a feed rate of 76.9 gr/h of feed sent in the empty reactor tube. The reactor inlet temperature was 575° C. The pressure was 1.5 bara. The composition of the effluent was analysed. The olefin content with respect to time is summarised in FIG. 28. It may be seen that substantially no olefins were produced by thermal cracking. This shows that catalytic cracking of the paraffins occurs in accordance with the invention.

1-11. (canceled)

12. A process for converting a hydrocarbon feedstock to provide an effluent containing light olefins comprising:
passing a hydrocarbon feedstock containing at least 25 wt. % C₅₊ paraffins through a reactor containing a crystalline silicate catalyst to produce an effluent including propylene.

13. The process of claim 12 further comprising forming the hydrocarbon feedstock by adding at least one C₆₊ paraffin to a C₄+ hydrocarbon feedstock cut comprising C₄₊ paraffins.

14. The process of claim 13, wherein the at least one C₆₊ paraffin comprises at least one C₆₋₂₀ linear paraffin.

15. The process of claim 13, wherein the hydrocarbon feedstock comprises from 1 to 80 wt. % of the at least one C₆₊ paraffin and from 20 to 99 wt. % of the C₄₊ hydrocarbon feedstock cut comprising C₄₊ paraffins.

16. The process of claim 12 wherein the hydrocarbon feedstock contains at least one C₄₊ olefin.

17. The process of claim 12, wherein the crystalline silicate is an MFI-type or MEL-type crystalline silicate having a silicon/aluminium atomic ratio of from 120 to 1000.

18. The process of claim 17, wherein the MFI-type crystalline silicate catalyst comprises silicalite.

19. The process of claim 12, wherein the hydrocarbon feedstock is passed over the crystalline silicate at a reactor inlet temperature of from 500 to 600° C.

20. The process of claim 12, wherein the hydrocarbon feedstock is passed over the crystalline silicate at a liquid hourly space velocity (LHSV) of from 5 to 30 h¹.

21. The process of claim 12, wherein the hydrocarbon feedstock is passed over the crystalline silicate at a pressure of from 0 to 2 barg.
